(19) United States
(12) Patent Application Publication
(10) Pub. No.: US 2026/0284090 A1
(43) Pub. Date: Sep. 24, 2026

(54) LIPOSOME COMPRISING PHOSPHATIDYLSERINE, PHOSPHATIDIC ACID AND OPTIONALLY CHOLESTEROL, COMPOSITION AND MEDICAL USE

(71) Applicant: BIOLT S.R.L.S., ROMA (IT)

(72) Inventors: Maurizio Fraziano, ROMA (IT); Noemi Poerio, ROMA (IT); Tommaso Olimpieri, ROMA (IT)

(73) Assignee: BIOLT S.R.L.S., ROMA (IT)

(21) Appl. No.: 18/996,072

(22) PCT Filed: Jul. 31, 2023
(86) PCT No.: PCT/IB2023/057747

§ 371 Date: Jan. 17, 2025

(30) Foreign Application Priority Data
Aug. 2, 2022 (IT) 102022000016401

(51) Int. Cl. A61K 31/7036; A61K 9/127

(52) U.S. Cl. CPC A61K 31/7036; A61K 9/127

(57) ABSTRACT

The present invention relates to a liposome consisting of an outer leaflet comprising phosphatidylserine, and an inner leaflet comprising phosphatidylserine and phosphatidic acid, characterized in that the zeta potential of said liposome is between −25 mV and −100 mV, and the molar ratio between the total phosphatidylserine and the phosphatidic acid is between 5:1 and 35:1, more preferably between 16:1 and 18:1, even more preferably 17:1. The present invention also concerns the use of said liposome and/or of a composition which comprises it in the treatment of bacterial infections, preferably in the treatment of infections caused by mycobacteria, in particular nontuberculous mycobacteria.

### FIELD OF THE INVENTION

The present invention is related to a liposome consisting of an outer leaflet comprising phosphatidylserine, and an inner leaflet comprising phosphatidylserine and phosphatidic acid, characterized in that the zeta potential of said liposome is between −25 mV and −100 mV, and the molar ratio between the total phosphatidylserine and the phosphatidic acid is between 5:1 and 35:1, more preferably between 16:1 and 18:1, even more preferably 17:1.

The present invention also concerns the use of said liposome and/or of a composition which comprises it in the treatment of bacterial infections, preferably in the treatment of infections caused by mycobacteria, in particular nontuberculous mycobacteria.

### STATE OF THE ART

Mycobacteria are a genus of Gram-variable bacilli, the only genus of the family Mycobacteriaceae capable of causing various diseases in humans.

Mycobacteria have a cell wall characterized by a thin layer of peptidoglycan bound to arabinogalactans, mycolic acids and phenolic glycolipids. Such complex cell wall gives mycobacteria the advantage of being completely impermeable to some of the substances most used in medical therapy, which partly explains the known resistance of mycobacteria to commonly used antibiotics.

In medicine, mycobacteria are traditionally divided into 3 groups.

A first group comprises tuberculous mycobacteria, i.e. capable of triggering tuberculosis in the animal host. This group consists of bacteria of the so-called Mycobacterium tuberculosis complex: Mycobacterium tuberculosis (responsible for human tuberculosis), Mycobacterium africanum (has a degree of infectivity of the organism similar to that of the Mycobacterium tuberculosis, but is less likely to cause disease) and Mycobacterium bovis (responsible for bovine tuberculosis, a zoonosis transmissible to humans via food).

A second group is consisting of nontuberculous mycobacteria, i.e. of mycobacteria which cause a range of pathologies other than tuberculosis in the human host, but only in concomitance with particular conditions that lower the immune defenses of the colonized organism (they are therefore opportunistic pathogens).

The third group is represented by the Mycobacterium leprae, the etiological agent of leprosy, which, although assimilable to the nontuberculous mycobacteria group, has peculiar clinical and biological characteristics.

Nontuberculous mycobacteria are a heterogeneous group of bacteria consisting of more than 150 different species, some of which are capable of infecting and causing diseases in humans other than tuberculosis.

It is known that most subjects infected by nontuberculous mycobacteria will not develop the clinical signs of infection.

It has been observed that the subjects most susceptible to infections by nontuberculous mycobacteria are those characterized by a weakening of the immune system, such as AIDS patients or patients undergoing transplants or people with lung lesions due to smoking or a previous tuberculosis, or affected by lung diseases such as emphysema, COPD or cystic fibrosis.

Infections caused by nontuberculous mycobacteria can be difficult to treat and require a long time to be eradicated, due to the resistance of these organisms to commonly prescribed antibiotics for the treatment of bacterial infections.

Among the species of nontuberculous mycobacteria, infections caused by Mycobacterium abscessus are more difficult to treat due to the resistance to commonly used antimicrobial drugs.

M. abscessus owes its antibiotic resistance to numerous mechanisms, among which an intrinsic drug resistance, a low permeability of the cell wall, the induction of drug efflux pumps, the presence of mutations in mycobacterial enzymes that do not convert prodrugs into active metabolites and/or the expression of numerous enzymes that can neutralize drugs or modify their specific targets.

M. abscessus can cause lung diseases, skin infections, central nervous system infections, bacteremia, eye infections and other less common diseases, which become very serious in immunocompromised patients.

Furthermore, in particular, M. abscessus can cause chronic lung disease in vulnerable hosts with underlying lung diseases such as cystic fibrosis, bronchiectasis and/or previous tuberculosis.

M. abscessus can also cause skin infections in immunodeficient patients, in patients who have recently undergone a surgical intervention, tattooing or acupuncture.

The incidence of infections by M. abscessus seems to increase over time (Lee et al., Mycobacterium abscessus Complex Infections in Humans, Emerg Infect Dis. 2015 Sep. 21 (9): 1638-46) and of particular concern are the outbreaks of nosocomial infections by M. abscessus reported among immunologically fragile patients.

In light of the above, there is a clear need to provide new products that have antimicrobial efficacy against antibiotic-resistant bacteria, in particular against mycobacteria, preferably nontuberculous mycobacteria.

A useful product for the treatment of lung infections caused by bacteria and/or viruses, in particular caused by mycobacteria, is the liposome described in WO2009/011007.

The liposome described in WO2009/011007 is an asymmetric liposome comprising phosphatidylserine molecules in the outer lipid layer and at least one bioactive lipid in the inner lipid layer. The bioactive lipid present in the inner lipid layer of the liposome is selected from the group consisting of phosphatidic acid, lysophosphatidic acid, arachidonic acid, sphingomyelin, sphingosine, sphingosine 1-phosphate, ceramide, leukotrienes, prostanoids, cyclopentenone prostaglandins or their derivatives.

However, this liposome, characterized by an absolute zeta potential value of less than 25 mV, appears to possess high instability.

It is known that the measurement of the zeta potential serves to predict the stability of electrostatic dispersions or interactions. In particular, the zeta potential is the potential generated as a result of the formation of a double electrical layer.

In fact, when moving in solution (Brownian motion), particles move together with an ionic double layer.

A high absolute zeta potential value (i.e. less than −30 mV and greater than +30 mV) causes the nanoparticles to remain far from each other, rebounding enough to eliminate the possibility of agglomeration, aggregation and/or flocculation.

A low zeta potential value, such as the one of the liposomes described in WO2009/011007, is responsible for particle aggregation and flocculation.

In fact, a liposome characterized by an absolute zeta potential value less than 25 mV is extremely unstable and tends to form aggregates when it is in solution.

In addition to this, the industrial process of obtaining the liposomes described in WO2009/011007, characterized by such high instability, poses quite a few difficulties and is not easily scalable.

Furthermore, the asymmetric liposomes described in WO2009/011007 have a high polydispersity index with the presence of two or more particle populations of different sizes. It is therefore evident that such asymmetric liposome is characterized by high size inhomogeneity.

Therefore, it is necessary to overcome the drawbacks of the known technique by providing a product, in particular a liposome, with antimicrobial activity against antibiotic-resistant bacteria that has greater stability, greater size homogeneity and that can be obtained through a scalable process.

### SUMMARY OF THE INVENTION

The Applicant proposed to solve the technical problem of the low stability of liposomes with antimicrobial activity of prior art by providing a liposome characterized by a zeta potential between −25 mV and −100 mV.

With respect to the asymmetric liposome of the prior art with an absolute zeta potential value lower than 25 mV, the liposome according to the invention is more stable, does not undergo flocculation and aggregation phenomena and is characterized by a greater size homogeneity.

Further than being particularly stable, the liposome according to the invention is characterized by a high antimicrobial activity as it is able to stimulate the immune system's response against one or more pathogens. In fact, as shown in the experimental section, the liposome of the invention enhances the microbicidal response of macrophages by directly transporting within them lipid intermediates (fatty acids, phospholipids, etc.), known to be involved in antibacterial activities (via phagolysosome biogenesis) or in the antiviral response.

According to the invention, these lipid intermediates are transported into macrophages via a liposome characterized by the presence of phosphatidylserine on the outer leaflet.

In fact, the presence of phosphatidylserine in the outer leaflet makes liposomes according to the invention similar to apoptotic bodies. In particular, apoptotic bodies are phagocytized by macrophages and also by target cells of viral and bacterial lung infections that are used to recognize and phagocytize apoptotic bodies, i.e. fibroblasts, epithelial and endothelial cells.

Once phagocytized by said cells, the liposome according to the invention releases bioactive lipids into them, which are known to be involved in antibacterial activities.

In detail, the bioactive lipids are involved in all the steps of the phagocytosis process, starting from the internalization of the pathogen to the maturation of the phagolysosome, and in the activation of the bactericidal response of the cells of the innate immune system, by recruiting, retaining and adjusting the activity of specific proteins involved.

Furthermore, advantageously, the phagocytosis of the liposome by macrophages is associated with the production of anti-inflammatory cytokines (Hoffmann P. R et al. J. Immunol. 2005; 174:1393-1404), which reduce the tissue-damaging inflammatory response.

Therefore, a first aspect of the present invention is a liposome consisting of an outer leaflet comprising phosphatidylserine, and an inner leaflet comprising phosphatidylserine and phosphatidic acid, characterized in that the zeta potential of said liposome is between −25 mV and −100 mV, and the molar ratio between the total phosphatidylserine and the phosphatidic acid is between 5:1 and 35:1.

Preferably, in the liposome according to the invention the molar ratio between the total phosphatidylserine and the phosphatidic acid is between 16:1 and 18:1, even more preferably is 17:1.

According to a preferred aspect, as shown in the experimental section, when the phosphatidylserine, in particular L-α-phosphatidylserine, and the phosphatidic acid, in particular L-α-phosphatidic acid, are in a molar ratio of 17:1 (intended as molar ratio between the total phosphatidylserine and the phosphatidic acid) the efficacy in the antimicrobial response towards the Mycobacterium abscessus is increased.

Therefore, in a particularly preferred embodiment, the liposome of the invention comprises phosphatidylserine and phosphatidic acid in a molar ratio of 17:1.

Optionally, the liposome according to the invention can comprise furthermore cholesterol, which may be arranged on the outer leaflet, on the inner leaflet, or on both of them.

In an embodiment, the liposome according to the first aspect of the invention incorporates one or more antibiotics, for example selected from first-line antitubercular antibiotics; second-line antitubercular antibiotics; aminoglycosides; glycylcyclines; penicillins; carbapenems; monobactams; quinolones; oxazolidinones; and macrolides.

The liposome according to the invention can be produced through the lipid film hydration method (example 1) or, preferably, by means of the microfluidic hydrodynamic focusing technique (example 2). Therefore, the present invention relates to a liposome as above described produced through the lipid film hydration method or through the microfluidic hydrodynamic focusing technique.

A second aspect of the present invention refers to a pharmaceutical composition comprising at least one liposome according to the first aspect of the invention, together with one or more pharmaceutically acceptable excipients and/or adjuvants.

A third aspect of the present invention refers to the use of the liposome as above defined and/or of a pharmaceutical composition which comprises it in the treatment of bacterial infections, in particular caused by mycobacteria, preferably nontuberculous mycobacteria.

As shown in the experimental section, the liposome according to the invention has shown particular efficacy against the Mycobacterium abscessus, opportunistic pathogen characterized by extensive and intrinsic antibiotic resistance.

Therefore, in a particularly preferred embodiment, said liposome and/or the composition which comprises it is used for the treatment of infections caused by Mycobacterium abscessus.

The last aspect of the present invention is a kit comprising the pharmaceutical composition as above defined, and one or more containers.

### DETAILED DESCRIPTION OF THE INVENTION

Object of the present invention is a liposome consisting of an outer leaflet comprising phosphatidylserine, and an inner leaflet comprising phosphatidylserine and phosphatidic acid, characterized in that the zeta potential of said liposome is between −25 mV and −100 mV, and the molar ratio between the total phosphatidylserine and the phosphatidic acid is between 5:1 and 35:1.

Liposomes are closed vesicular structures composed of a double-layer membrane consisting of phospholipids.

Phospholipids are amphiphilic molecules, in particular a class of lipids which have a phosphate-based hydrophilic polar head and a hydrophobic apolar tail.

On the biological level, phospholipids participate in the structure of cell membranes. Liposomes were discovered, quite by chance, in the early 1960s by the British hematologist Alec Bangham during the experimentation of an electronic microscope.

Since then, liposomes have been widely used as delivery systems for poorly absorbable or poorly soluble active substances, including pharmacological substances.

In fact, thanks to the tenside nature of phospholipids constituting them, liposomes “encapsulate” one or more active substances by favoring their release and promoting their absorption at mucosal level.

Secondly, the constituents of the phospholipid layers of the liposome are biocompatible and, furthermore, the liposome causes no side effects.

In addition, a further advantage results from the fact that the substances carried by the liposome are protected from the action of enzymes (proteases, nucleases) or from denaturing environments (pH). This further allows to preserve the integrity of the active substances encapsulated in the liposomes.

Finally, liposomes are biodegradable and are currently preparable on a large scale according to known techniques.

According to a preferred aspect, the liposome according to the invention comprises phosphatidylserine, in particular the L-α-phosphatidylserine, and the phosphatidic acid, in particular the L-α-phosphatidic acid, in a molar ratio between 16:1 and 18:1, more preferably 17:1. In particular, said molar ratio is the molar ratio between the total phosphatidylserine and the phosphatidic acid.

In the present invention, with the expression “total phosphatidylserine” it is intended to indicate the amount of phosphatidylserine resulting from the sum of the phosphatidylserine present on the outer leaflet of the liposome and of the phosphatidylserine present on the inner leaflet of said liposome.

Phosphatidylserine (in the present description also abbreviated as “PS”) is a phospholipid component of the plasma membrane of cells that plays a key role in the signaling of apoptosis. The phosphatidylserine in the healthy cell is normally exposed on the cytoplasmic side, whereas when the phosphatidylserine is exposed by flippases on the outer leaflet of the plasma membrane (“eat me” signal), it causes the recognition of the cell by macrophages and its subsequent elimination.

The presence of the phosphatidylserine in the outer leaflet of the liposome according to the invention makes such a liposome similar to an apoptotic body.

In biology, the term “apoptotic body” it is intended to indicate a vesicle resulting from the fragmentation of the nucleus and cytoplasm of a cell during a process of apoptosis (a form of programmed cell death), which is subsequently phagocytized by macrophages. In this manner, the liposome according to the invention will be recognized as an apoptotic body by macrophages and, possibly, by target cells of bacterial infections (e.g. fibroblasts, epithelial and endothelial cells) and phagocytized.

Advantageously, the phagocytosis of the liposome according to the invention allows the delivery of bioactive lipids involved in the antibacterial response. In particular, these lipids determine the formation and fate of the phagosome by influencing the curvature of the cell membrane wherein they are incorporated, contribute to membrane surface charge, and coordinate mechanisms of protein recruitment and association with key proteins in phagocytosis.

Furthermore, the phagocytosis of the liposome is associated with the production of anti-inflammatory cytokines, and is therefore responsible for reducing the tissue-damaging inflammatory response.

According to the invention, the phosphatidylserine is comprised in both the outer leaflet and the inner leaflet of the liposome.

In the present invention, the terms “comprises”, “comprised” and “comprising” shall be understood as open terms, which do not exclude the presence of compounds other than those mentioned.

In particular, the presence of these terms referred to the outer leaflet and the inner leaflet of the liposome indicates that, in addition to the phosphatidylserine and the phosphatidic acid, additional molecules may be present in both the outer leaflet and the inner leaflet of the liposome of the invention.

In an embodiment, the outer leaflet of the liposome according to the invention comprises phosphatidylserine and one or more further phospholipids.

Preferably, said further phospholipid which can be present in the outer leaflet of the liposome is the phosphatidic acid.

Phosphatidic acid (in the present invention shortened as “PA”) is a phosphoglyceride formally produced by the esterification of glycerol in position 1 and 2 with fatty acids and in position 3 with orthophosphoric acid.

According to the invention, the molar ratio between the total phosphatidylserine and the phosphatidic acid in the liposome is between 5:1 and 35:1.

In a particularly preferred embodiment, the molar ratio between the total phosphatidylserine present in the liposome and the phosphatidic acid is between 16:1 and 18:1, more preferably is 17:1.

In fact, as shown in the experimental section, the liposome comprising a molar ratio of 17:1 between the total phosphatidylserine and the phosphatidic acid has given the best results of intracellular inhibition of Mycobacterium abscessus with respect to liposomes having different ratios between phosphatidylserine and phosphatidic acid.

Furthermore, said liposome comprising a molar ratio of 17:1 between the total phosphatidylserine and the phosphatidic acid significantly increases the intracellular killing of Mycobacterium abscessus (see FIGS. 1A and B).

Optionally, the liposome according to the invention can comprise furthermore cholesterol, which can arrange on the outer leaflet, on the inner leaflet, or on both of them.

The cholesterol (in the present invention shortened by the acronym “CHO”) is an organic molecule belonging to the class of lipids and, more in detail, sterols.

The cholesterol is a polycyclic aliphatic alcohol, brute formula C27H460, consisting of the peri-hydro-1,2-cyclopentane-phenanthrene nucleus (tetracyclic nucleus typical of steroids), with a double bond in C5 and an isooctyl side chain in C17. The ending -ol derives from the fact that on the C3 of the first ring of carbon atoms (ring A) is present the hydroxyl group-OH.

From a biological point of view, the cholesterol has a structural function in the cell membrane: it increases the flexibility and stability of the double layer and keeps its fluidity, even at low temperatures. The cholesterol is inserted perpendicularly into the cell membrane, so that its hydroxyl group is located outwards, while the octyl chain is inserted deep into the membrane.

In the liposome according to the invention, the cholesterol can be optionally present.

In particular, the cholesterol can be optionally present both in the outer leaflet and in the inner leaflet of the liposome.

In a particularly preferred embodiment, the molar ratio between total phosphatidylserine, phosphatidic acid and cholesterol in the liposome according to the invention is 16.5:1:7.5.

In the liposome according to the present invention phospholipids of the liposome, mainly phosphatidylserine and phosphatidic acid, have a structural and bioactive role as above defined while the cholesterol has a mainly structural role.

The liposome so far described can advantageously be used as a delivery system for one or more drugs, for example drugs commonly used for the treatment of bacterial infections.

Therefore, in an embodiment, said liposome can encapsulate within it one or more antibiotics, for example first-line antitubercular antibiotics, second-line antitubercular antibiotics, aminoglycosides, glycylcyclines, tetracyclines, cephalosporins, penicillins, carbapenems, monobactams, quinolones, oxazolidinones, and macrolides.

In an embodiment, the liposome embeds within it first-line antitubercular antibiotics, preferably selected from isoniazid, rifampin, rifabutin, ethambutol, pyrazinamide, and streptomycin; second-line antitubercular antibiotics, preferably selected from cycloserine ethionamide, levofloxacin, moxifloxacin, ciprofloxacin, gatifloxacin acid, paraminosalicylic acid, kanamycin, and capreomycin; aminoglycosides, preferably selected from gentamicin and amikacin; glycylcyclines, preferably tigecycline; tetracyclines, preferably minocycline; cephalosporins, preferably selected from cefoxitin and ceftobiprole medocaryl sodium; penicillins, preferably amoxicillin; carbapenems, preferably imipenem; monobactams, preferably aztreonam lysine salt; quinolones, preferably moxifloxacin; oxazolidinones preferably linezolid; macrolides preferably selected from azithromycin, clarithromycin, erythromycin and fidaxomycin.

The liposome according to the invention can have sizes comprised between 30 nm and 350 nm, preferably between 50 nm and 300 nm, even more preferably between 100 nm and 250 nm.

The polydispersity index of the liposome of the present invention is between 0.10 and 0.55, preferably between 0.20 and 0.40, even more preferably between 0.25 and 0.35.

The zeta potential of the liposome of the present invention is lower than −25 mV, preferably lower than −30 mV, more preferably lower than −35 mV, and is greater than −100 mV, preferably greater than −70 mV, more preferably greater than −50 mV.

Preferably, the liposome according to the invention has a zeta potential between −25 mV and −85 mV, more preferably between −30 mV and −75 mV, even more preferably between −35 mV and −50 mV. The above zeta potential values are indicative of a good stability of the liposome that will not tend to form aggregates in solution, as the nanoparticles remain far enough away from each other, repelling enough to eliminate the possibility of agglomeration, aggregation and/or flocculation. The liposome object of the invention is produced through the lipid film hydration method (see example 1 and results of example 1) or, more preferably through the microfluidic hydrodynamic focusing technique (see example 2 and results of example 2).

In particular, the microfluidic hydrodynamic focusing technique (microfluidic hydrodynamic focusing, MHF) is based on the use of devices (chips) having a cross-flow geometry. Typically, a lipid stream in alcohol solution is forced to flow in the central (or inner) channel of the device. The lipid flow is intersected and covered by two lateral (or coaxial) flows of an aqueous phase (typically distilled water or aqueous buffers). In this way, the lipid-containing flow is hydrodynamically focused into a thin sheet with a rectangular section. In particular, the size of the focalized flow can be adjusted by modulating the flow rate ratio (Flow Rate Ratio-FRR) between the flows of aqueous and lipid phase, and the total flow rate (Total Flow Rate-TFR). The formation of liposomes in MHF chips is adjusted by the diffusion of various molecular species (mainly alcohol and water, but also lipids) at the liquid interface between the solvent (alcohol) and non-solvent (water) phases. The alcohol wherein the lipids are initially solubilized diffuses into the water (and simultaneously the water diffuses into the alcohol) until the alcohol concentration falls below the lipid solubility limit.

Therefore, alcohol diffusion triggers the formation of liposomes by means of a mechanism described as “self-assembly”. Specifically, the mutual diffusion of alcohol and water across the focused interface is believed to cause lipid precipitation, with the subsequent formation of intermediate structures, which subsequently close in on themselves, forming liposomal vesicles.

The microfluidic hydrodynamic focusing technique results to be particularly advantageous for the production of liposomes according to the invention as the sizes of liposomes can be controlled by adjusting the FRR flow ratio. As it is evident from tables 3a and 3b, the liposomes generated through the microfluidic hydrodynamic focusing technique have a greater homogeneity. A second aspect of the present invention relates to a pharmaceutical composition which comprises at least one liposome as above defined, and at least one pharmaceutically acceptable excipient.

The composition of the invention can comprise at least one antibiotic, eventually embedded in the liposome, and/or outside it.

In particular, in an embodiment of the composition of the invention, the at least one antibiotic is present within the liposome, encapsulated in it.

In another embodiment, the at least one antibiotic is present in the pharmaceutical composition of the invention outside the liposomes.

In a further embodiment, the at least one antibiotic is present in the composition, both inside and outside the liposomes.

When the at least one antibiotic is present in the composition both inside the liposomes, and outside the liposomes, said antibiotic can be the same or a combination of two or more antibiotics.

As an example, antibiotics that can be present in the composition according to the invention are first-line antitubercular antibiotics, preferably selected from isoniazid, rifampin, rifabutin, ethambutol, pyrazinamide, and streptomycin; second-line antitubercular antibiotics, preferably selected from cycloserine ethionamide, levofloxacin, moxifloxacin, ciprofloxacin, gatifloxacin acid, paraminosalicylic acid, kanamycin, and capreomycin; aminoglycosides, preferably selected from gentamicin and amikacin; glycylcyclines, preferably tigecycline; tetracyclines, preferably minocycline; cephalosporins, preferably selected from cefoxitin and ceftobiprole medocaryl sodium; penicillins, preferably amoxicillin; carbapenems, preferably imipenem; monobactams, preferably aztreonam lysine salt; quinolones, preferably moxifloxacin; oxazolidinones preferably linezolid; macrolides preferably selected from azithromycin, clarithromycin, erythromycin and fidaxomycin.

The at least one pharmaceutically acceptable excipient present in the composition according to the invention can be excipients and/or adjuvants commonly present in formulations suitable for aerial, dermal, or mucosal (e.g. intestinal mucosa) administration.

In a preferred embodiment, the composition according to the invention comprises one or more pharmaceutically acceptable excipients and/or adjuvants for the administration by inhalation.

The liposome according to the invention proved to be particularly useful in the treatment of bacterial infections, in particular caused by nontuberculous mycobacteria.

Furthermore, a third aspect of the present invention refers to the use of the liposome as above defined and/or of a pharmaceutical composition comprising at least one of said liposomes in the treatment of bacterial infections.

In an embodiment, the liposome according to the invention and the composition which comprises it can be used for the treatment of bacterial infections caused by Pseudomonas aeruginosa, Enterococcus faecium, Staphylococcus aureus, Klebsiella pneumoniae, Acinetobacter baumannii, Enterobacter spp, Propionibacterium acnes, Mycobacterium tuberculosis complex, Mycobacterium tuberculosis, Mycobacterium africanum, Mycobacterium bovis, Mycobacterium ulcerans, Mycobacterium leprae, Mycobacterium abscessus, Mycobacterium avium complex (MAC), Mycobacterium chimaera, Mycobacterium kansasii, Mycobacterium bolletii, Mycobacterium massiliense and Mycobacterium abscessus, preferably by Mycobacterium abscessus.

The release of bioactive lipids by the liposome of the invention in macrophages that incorporate it generally allows to enhance the antimicrobial response. In fact, the lipids released by the liposome of the invention (recognized by macrophages as an apoptotic body and incorporated into them) are involved in the internalization of the pathogen until the maturation of the phagolysosome.

As it is known, the phagolysosome is the vesicle formed within phagocytes (cells capable of performing phagocytosis and intracellular killing of pathogens, including macrophages) by the fusion of the phagosome (endocytic vesicle containing foreign material, e.g. the pathogen), with the lysosome (vesicle containing lysosomal enzymes capable of degrading the foreign material, e.g. the pathogen, contained within the phagosome).

It is obvious, therefore, that the enhancement of the antimicrobial response does not depend on the type of micro-organism causing the infection but on the particular mechanism by which the liposome according to the invention acts.

In a preferred embodiment, the liposome of the invention and the composition that comprises it are used for the treatment of infections caused by mycobacteria, preferably caused by nontuberculous mycobacteria.

Examples of mycobacteria against which the liposome according to the first aspect of the invention and the composition according to the second aspect of the invention are useful are Mycobacterium tuberculosis complex, Mycobacterium avium complex (MAC), Mycobacterium chimaera, Mycobacterium kansasii, Mycobacterium bolletii, Mycobacterium massiliense and Mycobacterium abscessus.

Nontuberculous mycobacteria can be classified into two groups: slow-growing and fast-growing. To the first group belong organisms such as Mycobacterium tuberculosis complex, Mycobacterium avium complex (MAC), M. chimaera and M. kansasii, while to the second one belong M. abscessus complex, of which M. abscessus ssp. abscessus, M. abscessus ssp. bolletii and M. abscessus ssp. massiliense.

In an embodiment, the liposome according to the first aspect of the invention and the composition according to the second aspect of the invention are used for the treatment of bacterial infections caused by Mycobacterium abscessus spp.

As shown in the experimental section, in the PSPA-L liposome with molar ratio between the total phosphatidylserine and the phosphatidic acid between 5:1 and 35:1, the replication of the Mycobacterium abscessus is decreased.

As it is obvious from FIG. 1 B), liposomes having a molar ratio between the total phosphatidylserine and the phosphatidic acid of 8.5:1 and of 34:1 show antimicrobial activity against M. abscessus and reduce the replication of M. abscessus within macrophages with respect to macrophages not treated with the liposomes according to the invention.

In an even more advantageous way, the liposome having a molar ratio between the total phosphatidylserine (sum of the PS present in the outer leaflet and in the inner leaflet of the liposome) and the phosphatidic acid of 17:1 enhances the antimicrobial response of macrophages to the Mycobacterium abscessus.

Therefore, preferably, the liposome of the invention and the composition which comprises it are used for the treatment of infections caused by Mycobacterium abscessus.

In particular, the composition and/or the liposome comprised in it can be used for the treatment of nontuberculous mycobacteria lung disease, a rather rare, but chronic and debilitating pathology, which leads to decrease of lung functionality.

It has been advantageously observed that a combined therapy based on antibiotics and bioactive liposomes, can be a valid strategy to simultaneously target the extracellular and intracellular pathogen, as on the one hand the liposome of the invention acts by favoring the activation of the bactericidal response of the cells of the innate immune system, by recruiting, retaining and adjusting the activity of specific proteins involved and, on the other hand, the antibiotic acts directly on the pathogen.

As reported in the experimental section, the treatment combined with the liposome comprising phosphatidylserine and phosphatidic acid in a molar ratio of 17:1 or with the liposome comprising phosphatidylserine, phosphatidic acid and cholesterol in a ratio di 16.5:1:7.5 and amikacin additively reduces the intracellular growth of Mab in human macrophages. In particular, the combined treatment with said liposomes has induced a significantly greater reduction in the Mab replication index with respect to single treatments (see FIG. 3).

A further object of the present invention is a kit comprising the pharmaceutical composition according to the second aspect of the invention, and one or more containers.

In an embodiment, the kit comprises a container for the pharmaceutical composition comprising one or more liposomes which incorporate within them an antibiotic.

In another embodiment, said kit comprises a container for the pharmaceutical composition comprising one or more liposomes which do not incorporate within them an antibiotic and one or more containers for antibiotics to be associated to the treatment with the liposome according to the invention.

In a further embodiment, said kit comprises a container for the pharmaceutical composition comprising one or more liposomes which incorporate within them an antibiotic and one or more containers for antibiotics to be associated to the treatment with the liposome according to the invention.

An aspect of the present description refers to a method for the treatment of one or more bacterial infections, preferably caused by mycobacteria, in particular nontuberculous mycobacteria, which comprises administering to a subject having one or more bacterial infections one or more of the liposomes according to the invention and/or the pharmaceutical composition according to the invention.

The effective dose and the regimen for administration of the liposome according to the invention and/or of a composition which comprises it depends on many factors, such as for example the administration way or the degree of affliction of the individual receiving the treatment.

Furthermore, both the effective dose and the regimen for the administration will be determined by the doctor where the subject receiving the treatment is being treated.

In a preferred embodiment, the method of treatment of one or more bacterial infections, preferably mycobacterial, comprises administering one or more liposomes as above defined and/or the composition which comprises them, by inhalation, more preferably by aerosol.

### DESCRIPTION OF FIGURES

In the figures attached to the present patent application and in the following examples are present the following abbreviations:
PS: phosphatidylserine;
PS-NBD: phosphatidylserine conjugated to NBD fluorochrome;
PA: phosphatidic acid;
CHO: cholesterol;
Mab: Mycobacterium abscessus;
Ctrl: control;
PSPA-L: liposome obtained through the lipid film hydration method comprising PS on the outer leaflet and PS and PA on the inner leaflet;
PSPACHO-L: liposome obtained through the lipid film hydration method comprising
PS on the outer leaflet and PS, PA and CHO on the inner leaflet;
PS/PA: asymmetric liposome of the prior art obtained through the Weitz method, as described in WO2009/011007A2, comprising PS exclusively on the outer leaflet and PA exclusively on the inner leaflet;
PSPA-M: liposome obtained through the microfluidic hydrodynamic focusing technique comprising PS on the outer leaflet and PS and PA on the inner leaflet;
AMK: amikacin;
dTHP-1: THP-1 cells induced to differentiate through stimulation with forbol-12-myristate-13-acetate (PMA) and then used as a model of human macrophages.

FIG. 1 In vitro comparison of the efficacy among various formulations of PSPA-L liposomes in terms of intracellular killing of Mab.

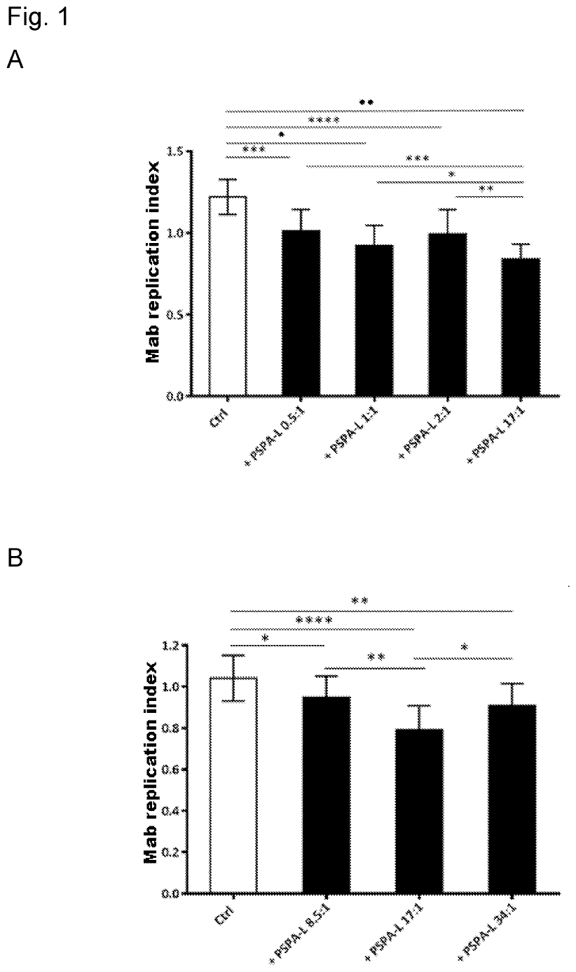

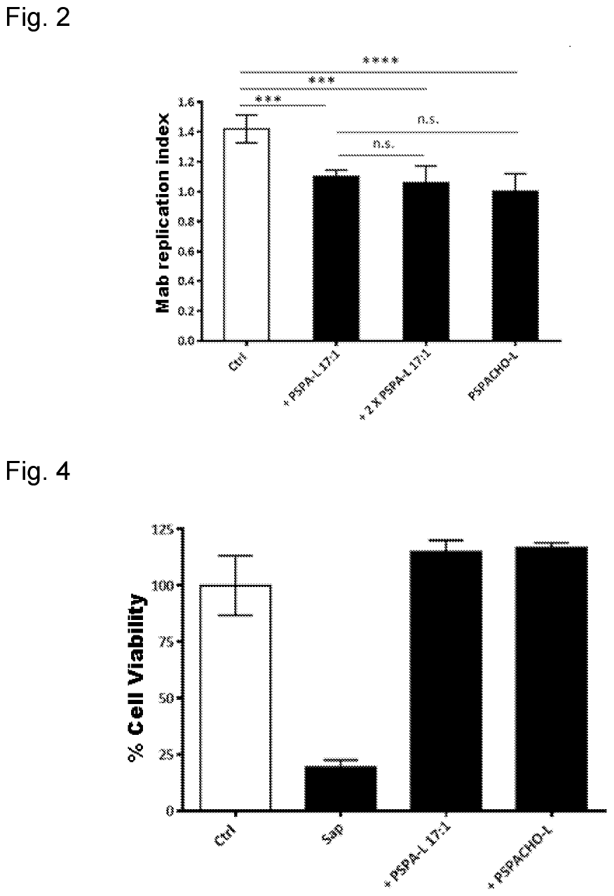

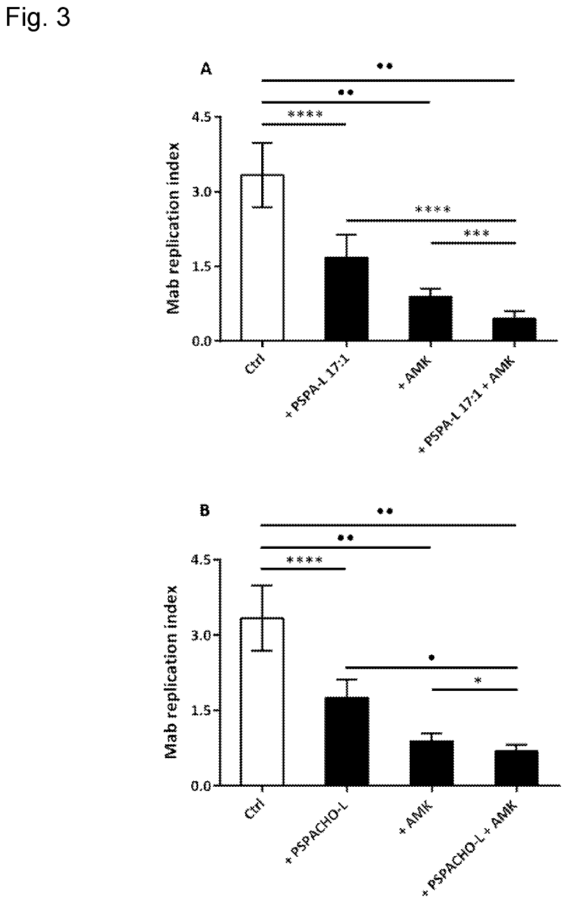

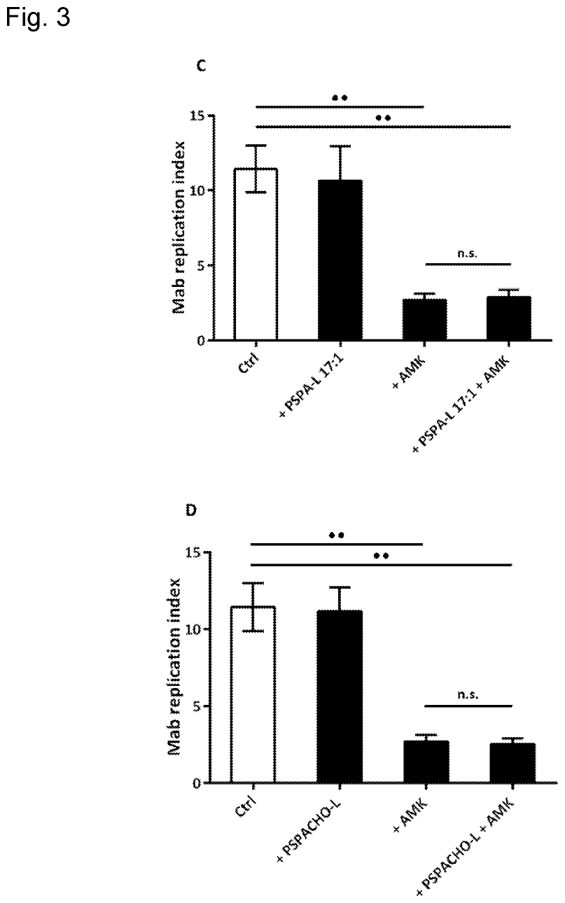

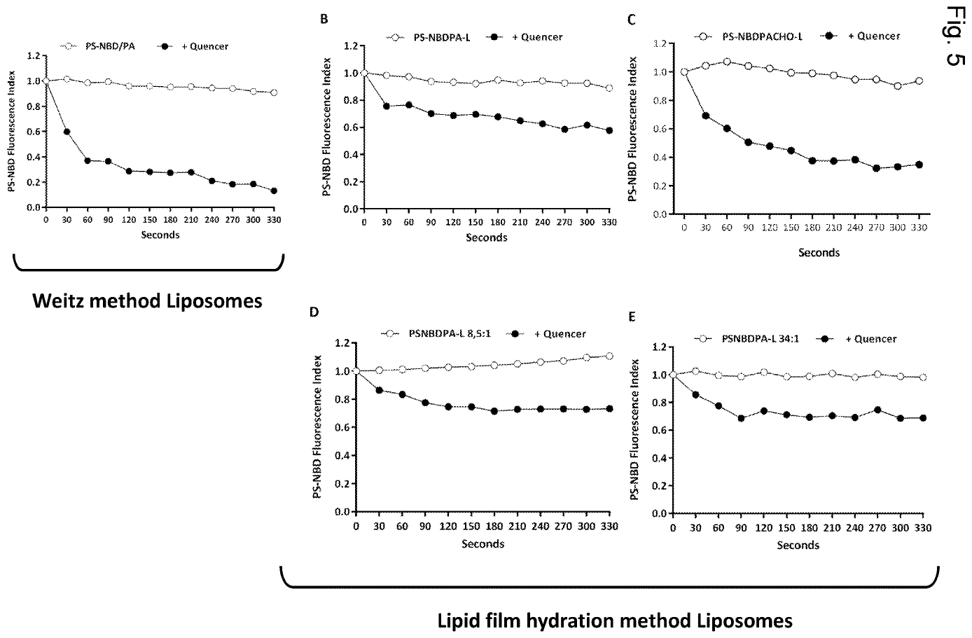

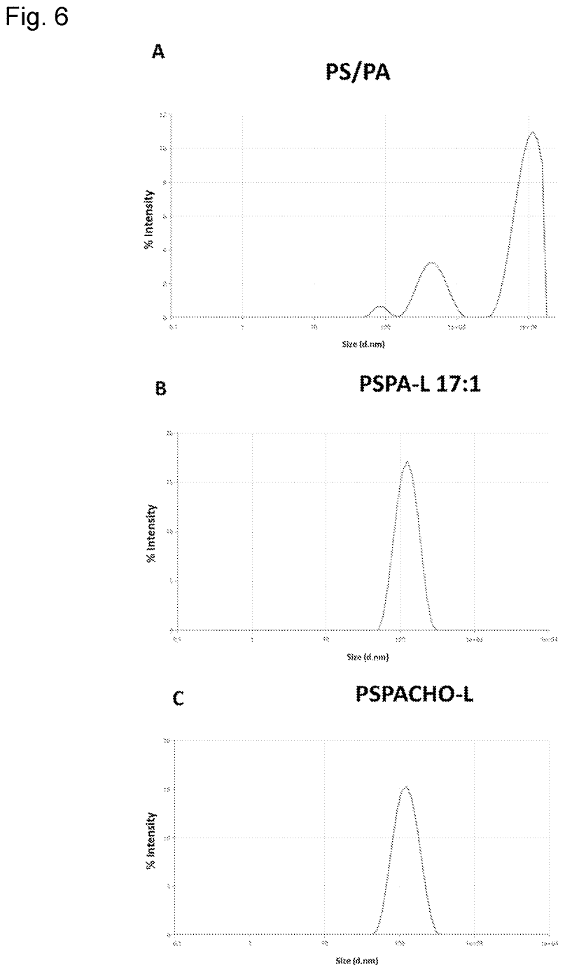

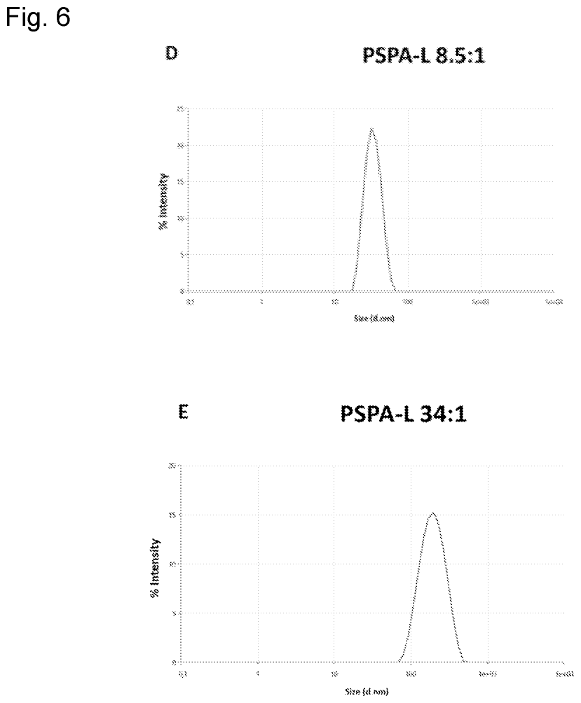

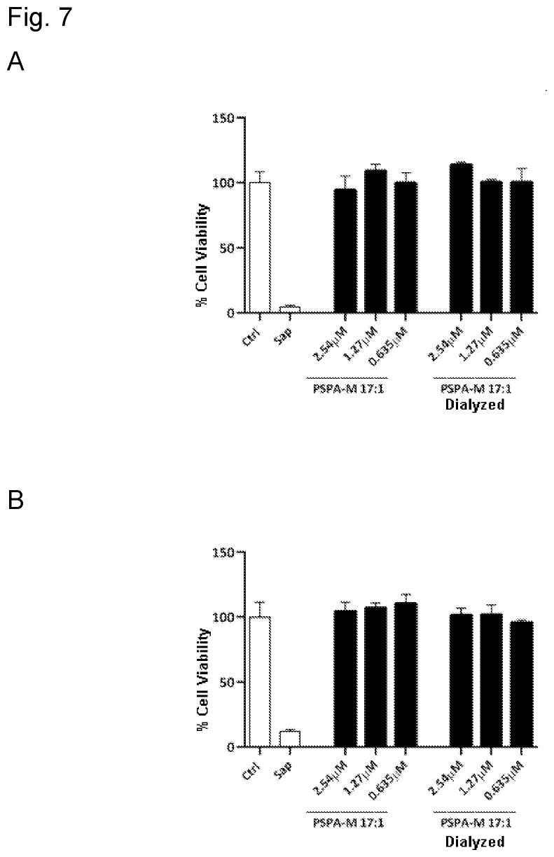

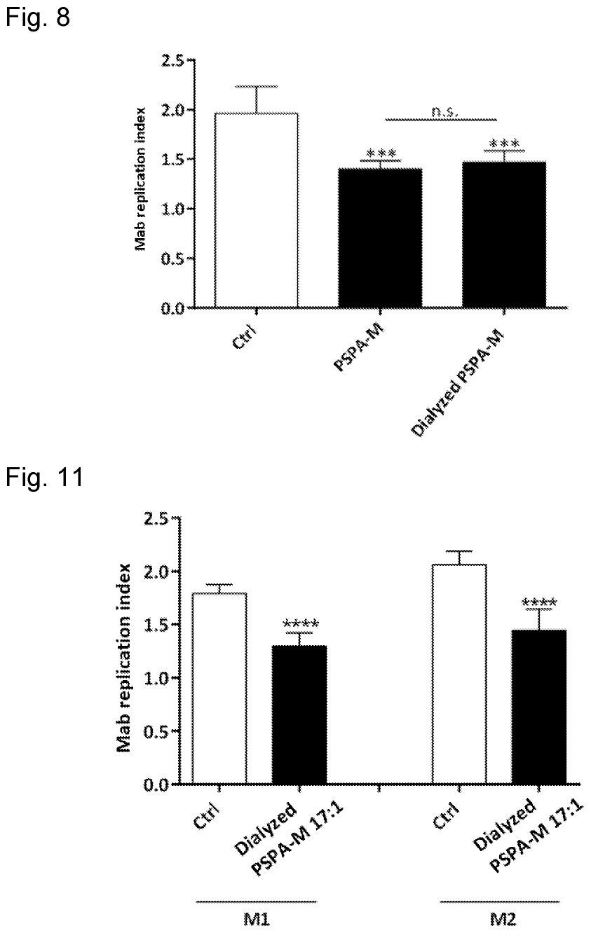

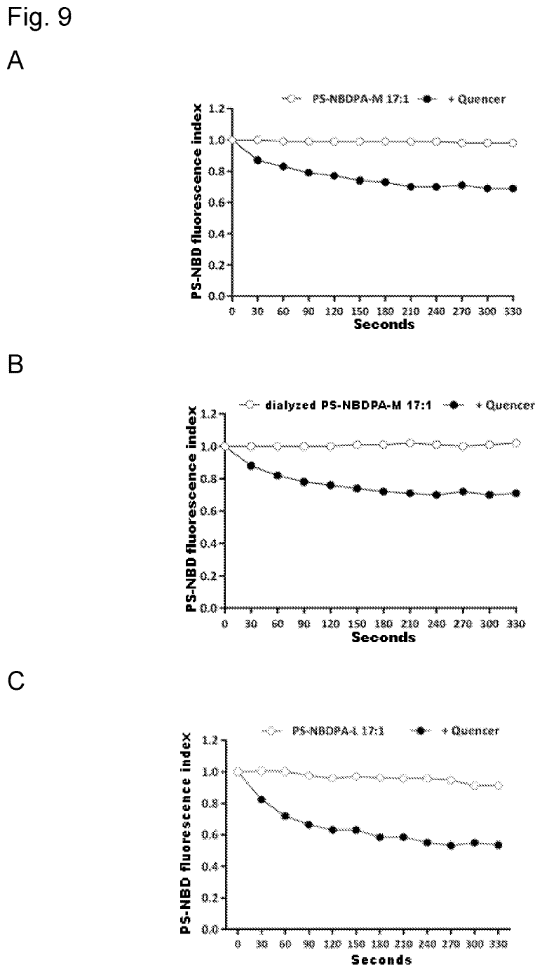

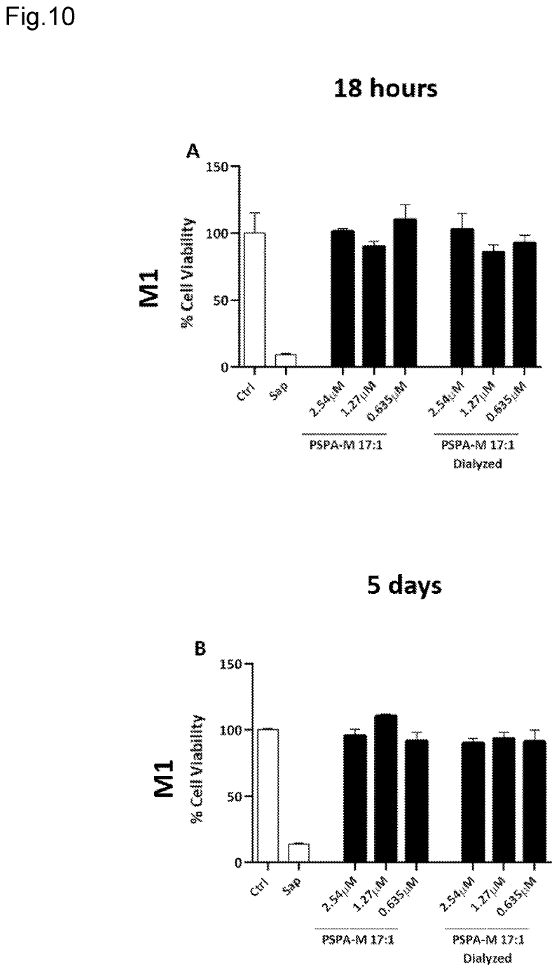

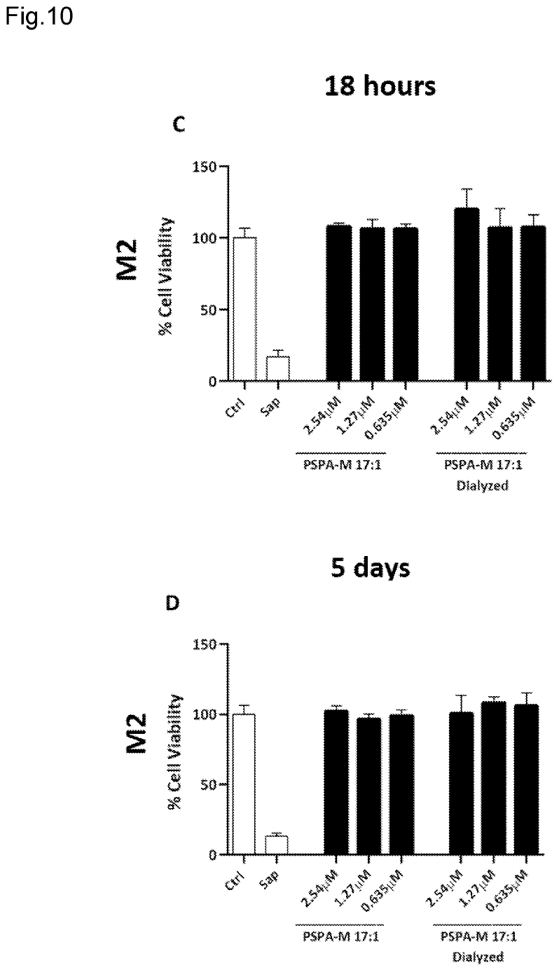

5×10⁵ dTHP-1 cells/well were seeded in 24 well-plates and were infected with the Mab reference strain (ATCC 19977) for 3 hours and then treated for 18 hours with PSPA-L in molar ratio: 0.5:1, 1:1, 2:1, 17:1 (FIG. 1A) and PSPA-L in molar ratio 8.5:1, 17:1, 34:1 (FIG. 1 B). The Mab bacterial growth was assessed by CFU assay. The replication index was calculated as the ratio between the CFUs obtained 18 hours after the infection, in the absence (control) or presence of liposomal formulations, and the CFUs obtained before the addition of liposomes. The results are shown as the average±standard deviation of the values obtained from the triplicate of each condition of two independent experiments. *p<0.05; **p<0.01; ***p<0.001; ****p<0.0001; *p<0.00001; **p<0.00001 from t test of Student with two-tail distribution.

FIG. 2 Assessment of the efficacy of PSPA-L 17:1 liposomes after addition of cholesterol and of dosage variation in terms of intracellular killing of Mab. 5×10⁵ dTHP-1 cells/well were seeded in 24 well-plates and were infected with the Mab reference strain (ATCC 19977) for 3 hours and then treated for 18 hours with PSPA-L molar ratio 17:1, PSPA-L molar ratio 17:1 in double dose (2×PSPA-L 17:1) or PSPACHO-L. The bacterial growth was assessed by CFU assay. The replication index was calculated as the ratio between the CFUs obtained 18 hours after the infection, in the absence (control) or presence of liposomal formulations, and the CFUs obtained before the addition of liposomes. The results are shown as the average±standard deviation of the values obtained from the triplicate of each condition. n.s. p=not significant; ***p<0.001; ****p<0.0001 from t test of Student with two-tail distribution.

FIG. 3 Assessment of the efficacy of the combined strategy consisting of the PSPA-L 17:1 or PSPACHO-L liposomes and amikacin in terms of extracellular and intracellular killing of Mab.

5×10⁵ dTHP-1 cells/well were seeded in 24 well-plates and were infected with the Mab reference strain (ATCC 19977) for 3 hours and then treated for 18 hours with PSPA-L molar ratio 17:1 or PSPACHO-L, and/or 4 μg/ml of amikacin (AMK) for further 18 hours. Finally, the supernatant was collected, the cells were lysed and both analyzed for the intracellular (A and B) and extracellular (C and D) bacterial growth. The replication index was calculated as the ratio between the CFUs obtained 18 hours after the infection, in the absence (control) or presence of liposomal formulations and/or AMK, and the CFUs obtained before the addition of liposomes. The results are shown as the average±standard deviation of the values obtained from the triplicate of each condition. *p<0.05; **p<0.01; ***p<0.001; ****p<0.0001; *p<0.00001; **p<0.00001 from t test of Student with two-tail distribution.

FIG. 4 Assessment of cell viability after treatment with PSPA-L 17:1 and PSPACHO-L liposomes.

2×10⁵ dTHP-1 cells/well were seeded in 96 well-plates and were incubated in complete medium (control, Ctrl) or treated for 18 hours with PSPA-L at molar ratio 17:1, PSPACHO-L or Saponin (Sap, used as a negative control of cell viability) and then the viability of dTHP1 cells was monitored with the MTT assay. The results are shown as the average±standard deviation of the percentage of cell viability of the triplicate of each condition.

FIG. 5 Comparison of the arrangement of PS in asymmetric PS/PA liposomes and PSPA-L liposomes.

The fluorescence intensity of the PS-NBD/PA (A), PS-NBDPA-L 17:1 (B), PS-NBDPACHO-L (C), PS-NBDPA-L 8.5:1 (D), PS-NBDPA-L 34:1 (E) liposomes was monitored in kinetics by taking 12 measurements every 30 seconds (for a total of 5 min), in the presence or absence of the quencher solution. The results are expressed as fluorescence intensity index calculated as the ratio between the 12 measurements over time (0, 30, 60, 90, 120, 150, 180, 210, 240, 270, 300, 330 seconds) and the measurement at time 0 seconds.

FIG. 6 Assessment of the size distribution of PSPA-L, PSPACHO-L and PS/PA liposomal formulations

Samples of the PS/PA (A), PSPA-L 17:1 (B), PSPACHO-L (C), PSPA-L 8.5:1 (D) and PSPA-L 34:1 (E) formulations were analyzed in terms of size distribution using Zeta-Sizer Advance Ultra-Malvern Panalytical. The graphs show a single batch acquisition and are representative of the triplicate acquisition of two separate batches of liposomal preparations. Batch 1 and batch 2 relative to the same liposomal preparation were produced at different times. The size distribution is represented as percentage intensity (% intensity) in relation to particle diameter in nanometers (d.nm).

FIG. 7 Assessment of cell viability after treatment with PSPA-M 17:1 liposomes

2×10⁵ dTHP-1 cells/well were seeded in 96 well-plates and were incubated in complete medium (control, Ctrl) or treated for 18 hours (FIG. 7A) or 5 days (FIG. 7B) with PSPA-M at molar ratio 17:1, dialyzed or non-dialyzed, at concentrations 2.54 μM, 1.27 μM, or 0.635 μM, or Saponin (Sap, used as a negative control of cell viability) and then the cell viability was monitored with the MTT assay. The results are shown as the average±standard deviation of the percentage of cell viability of the triplicate of each condition.

FIG. 8 Assessment of the efficacy of dialyzed or non-dialyzed PSPA-M 17:1 liposomes in terms of intracellular killing of Mab

5×10⁵ dTHP-1 cells/well were seeded in 24 well-plates and were infected with the Mab reference strain (ATCC 19977) for 3 hours and then treated for 18 hours with 1.27 μM PSPA-M molar ratio 17:1, dialyzed or non-dialyzed. The bacterial growth was assessed by CFU assay. The replication index was calculated as the ratio between the CFUs obtained 18 hours after the infection, in the absence (control) or presence of liposomal formulations, and the CFUs obtained before the addition of liposomes. The results are shown as the average±standard deviation of the values obtained from the triplicate of each condition. n.s. not significant and ***p<0.001 from t test of Student with two-tail distribution.

FIG. 9 Comparison of the arrangement of PS in dialyzed PSPA-M 17:1, PSPA-M 17:1 and PSPA-L liposomes

The fluorescence intensity of the dialyzed PS-NBDPA-M 17:1 (A), PS-NBDPA-M 17:1 (B) and PS-NBD-PA-L 17:1 (C) liposomes was monitored in kinetics by taking 12 measurements every 30 seconds (for a total of 5 min), in the presence or absence of the quencher solution. The results are expressed as fluorescence intensity index calculated as the ratio between the 12 measurements over time (0, 30, 60, 90, 120, 150, 180, 210, 240, 270, 300, 330 seconds) and the measurement at time 0 seconds.

FIG. 10 Assessment of cell viability of primary M1 or M2 macrophages after treatment with PSPA-M 17:1 liposomes

2×10⁵ M1 cells/well or M2/well, were seeded in 96 well-plates and were incubated in complete medium (control, Ctrl) or treated for 18 hours (A and C) or 5 days (B and D) with PSPA-M at molar ratio 17:1, dialyzed or non-dialyzed, at concentrations 2.54 μM, 1.27 μM, or 0.635 μM, or Saponin (Sap, used as a negative control of cell viability) and then the cell viability was monitored with the MTT assay. The results are shown as the average±standard deviation of the percentage of cell viability of the triplicate of each condition.

FIG. 11 Assessment of the efficacy of dialyzed or non-dialyzed PSPA-M 17:1 liposomes in terms of intracellular killing of Mab

5×10⁵ M1 cells/well or M2 cells/well were seeded in 24 well-plates and were infected with the Mab reference strain (ATCC 19977) for 3 hours and then treated for 18 hours with 1.27 μM dialyzed PSPA-M molar ratio 17:1. The bacterial growth was assessed by CFU assay. The replication index was calculated as the ratio between the CFUs obtained 18 hours after the infection, in the absence (control) or presence of liposomal formulations, and the CFUs obtained before the addition of liposomes. The results are shown as the average±standard deviation of the values obtained from the triplicate of each condition. ****p<0.0001 from t test of Student with two-tail distribution.

### EXAMPLES

### Example 1—Lipid Film Hydration Method

Preparation of Liposomal Formulations Having Different Molar Ratios Between Lipids and Study of their Efficacy Against Infection by Mycobacterium abscessus

### Materials and Methods

### Preparation of Liposomal Formulations

Using the lipid film hydration method, liposomal formulations consisting of L-α-phosphatidylserine (PS; Avanti Polar Lipids) and L-α-phosphatidic acid (PA; Avanti Polar Lipids) (PSPA-L), eventually with addition of cholesterol (CHO; Avanti Polar Lipids) (PSPACHO-L) were generated. In particular, 7 liposomal formulations having different molar ratios between lipids were produced, as indicated in table 1.

TABLE 1

| Liposomal | Molar ratio | Nanomoles |
|---|---|---|
| formulation | (PS:PA) | (PS:PA) |
| PSPA-L | 0.5:1 | 14.1:28.2 |
| | 1:1 | 21.2:21.2 |
| | 2:1 | 28.2:14.1 |
| | 8.5:1 | 38:4.4 |
| | 17:1 | 40:2.4 |
| | 34:1 | 41.2:1.2 |

| | Molar ratio | Nanomoles |
|---|---|---|
| | (PS:PA:CHO) | (PS:PA:CHO) |
| PSPACHO-L | 16.5:1:7.5 | 40:2.4:12.7 |

For each formulation, a total amount of PS and PA phospholipids equal to 42.4 nmoles in the reciprocal ratios indicated in Table 1 were placed in a glass tube, to which 12.7 nmoles CHO were eventually added, thus generating respectively PSPA-L and PSPACHO-L liposomes (Table 1). Lipids, previously dissolved in chloroform, were then dehydrated using Rotavapor R-100 (Buchi). After the evaporation of the chloroform, the lipid film was rehydrated with 1 ml saline solution (NaCl 0.9%). Formulations were then shaken for 10 minutes, and subsequently sonicated in a sonicator bath for 30 minutes in order to favor the formation of unilamellar vesicles.

Finally, liposomes were extruded with the mini extruder (Avanti Polar Lipids) using a membrane having a pore size equal to 200 nm (Avanti Polar Lipids).

The PS/PA liposomal formulation was generated with the Weitz method (Proc Natl Acad Sci USA. 2003. 100 (19): 10718-21), as described in WO2009/011007A2. Shortly, the lipid constituting the inner leaflet, L-α-phosphatidic acid (PA; Avanti Polar Lipids), was suspended in anhydrous dodecane (Sigma) at the concentration of 0.05 mg/ml. For the production of the outer leaflet, was used the L-α-phosphatidylserine (PS; Avanti Polar Lipids) that was added to a solution of dodecane: silicone 99:1 in order to obtain a final concentration of 0.05 mg/ml. Liposomes were prepared by adding 2 ml of the outer lipid leaflet suspension to 3 ml of saline solution (NaCl 0.9%). Finally, 100 μl of the inner lipid leaflet suspension was added to the 2 ml of outer lipid leaflet suspension and the samples were centrifuged at 120 g for 10 min. After the centrifugation, the aqueous phase containing the vesicles was collected using a 5 ml syringe with a 16-gauge stainless steel needle in order to produce outer PS/inner PA liposomes (PS/PA). Liposomes were then quantified with the FACSCalibur cytofluorometer (Becton Dickinson), allowing the quantification of monodisperse vesicles>0.2 μm in diameter.

### Cell Cultures

The monocyte/macrophage cell line THP-1 was provided by the European Collection of Authenticated Cell Culture (ECACC 88081201), and cultured in RPMI 1640 containing fetal bovine serum (10%), gentamicin (5 μg/ml), L-glutamine (2 mM), non-essential acidic amino acids (1 mM), sodium pyruvate (1 mM) in 75 cm2 polystyrene flasks. Before the experiments, the cells (5×105) were seeded in 24 or 96 well-plates and the cells were induced to differentiate through stimulation for 72 hours with forbol-12-myristate 13-acetate (PMA) (20 ng/ml) and used as a model of human macrophages (dTHP-1).

### Bacterial Cultures

The individual colonies of Mycobacterium abscessus (Mab) (ATCC® 19977) were obtained by seeding the mycobacterium on Middlebrook 7H10 (7H10-BD Difco™) solid medium integrated with oleic acid, albumin, dextrose and catalase (OADC), and then resuspended in 15 ml of Middlebrook 7H9 broth (7H9-BD Difco™) integrated with albumin, dextrose and catalase (ADC) and grown in Erlenmeyer flasks at 37° C. in agitation for 48 hours. The growth of bacterial cultures was monitored by measuring the optical density at a wavelength of 600 nm with a spectrophotometer (Varioskan LUX Multimode Microplate Reader, Thermo Fisher Scientific). The bacilli were stored at −80° C. until use after suspension in Microorganism Preservation System-Protect (Technical Service Consultants Ltd).

### Infection and Assessment of In Vitro Bacterial Intracellular Growth

In order to assess the intracellular bacterial growth, monocyte/macrophage dTHP-1 cells were distributed in 24 well-plates at a concentration of 5×10⁵ cells/ml. The cells were infected with Mab, for 3 hours at 37° C. at a MOI of 10, and after the infection the extracellular bacilli were killed through a 1-hour incubation with 250 μg/ml of amikacin. Cells were then washed and incubated for further 18 hours with: PSPA-L molar ratio 1:1, PSPA-L molar ratio 2:1, PSPA-L molar ratio 1:2, PSPA-L molar ratio 17:1, PSPA-L molar ratio 8.5:1, PSPA-L molar ratio 34:1 or PSPACHO-L molar ratio 16.5:1:7.5, whose concentration of phospholipids was equal to 1.27 μM. Finally, cells were lysed with 1% deoxycholate (Sigma), samples diluted in PBS-tween 80 and CFU quantified by plating the bacilli in triplicate on 7H10.

In order to assess the in vitro efficacy of a combined therapy on the viability of extracellular and intracellular mycobacteria, dTHP-1 cells were infected with Mab, at a MOI of 10 for 3 hours at 37° C. Cells were then stimulated with PSPA-L molar ratio 17:1 or PSPACHO-L molar ratio 16.5:1:7.5 and/or 4 μg/ml of amikacin (AMK) for 18 hours. Both the extracellular and the intracellular bacterial growth were assessed through CFU assay by plating the bacilli in triplicate on 7H10.

### MTT Assay for Assessment of Cell Viability

The cell viability of dTHP-1, previously stimulated for 24 hours with PSPA-L molar ratio 17:1 or PSPACHO-L molar ratio 16.5:1:7.5, whose concentration of phospholipids was equal to 1.27 μM was assessed by assay with 3-(4,5-dimethylthiazol-2yl)-2,5-diphenyl tetrazolium bromide (MTT). The assay with MTT (Molecolar Probe) is based on the intracellular reduction of tetrazolium salts by the dehydrogenase succinate mitochondrial enzyme (SDH) into crystals of a bluish product called formazan, a reduction that can only be produced in metabolically active cells whose optical density is measured by spectrophotometer using a wavelength of 540 nm. This assay was conducted according to the guidelines indicated in the user manual.

### Distribution of Phosphatidylserine on the Surface of Liposomes

The assessment of the PS arrangement was analyzed through the fluorimetric method previously described (Proc Natl Acad Sci USA. 2003. 100 (19): 10718-21). Shortly, PSPA-L with molar ratio 17:1, 8.5:1 and 34:1, PSPACHO-L and PS/PA liposomes were generated, as previously described in the paragraph “Preparation of liposomal formulations”, using a fluorescent analogue of PS [1-palmitoyl-2-{12-[(7-nitro-2-1,3-benzoxadiazol-4-yl)amino]dodecanoyl}-sn-glycero-3-phosphoserine, PS-NBD, AvantiPolar Lipids), in order to assess its presence on the outer surface of the liposomes. In particular, the fluorescence of PS-NBD was monitored before and after the addition of a quenching solution (1M sodium dithionite (Na2S204) in TES 5 mM a pH 9, daily prepared). The addition of the quencher to the vesicles in suspension only reduces the fluorescence of PS-NBD localized on the outer leaflet of the liposome, not being able to cross the lipid bilayer. Fluorescence measurement was performed in kinetics for 5 min every 30 seconds (12 measurements) with the Varioskan LUX multimode microplate reader fluorometer (Thermo Fisher Scientific) by setting the excitation and emission wavelengths to 470 nm and 550 nm respectively.

### DLS Characterization of Liposomes

Analyses were carried out on PSPA-L with molar ratio 8.5:1, 17:1, and 34:1, PSPACHO-L and PS/PA samples stored in a refrigerator at 4° C., using the Zeta-Sizer Advance Ultra-Malvern Panalytical, provided with a He—Ne laser (λ=633 nm) with a maximum power of 10 mW. Measurements were carried out at a constant temperature value of 25° C., with a scattering angle of 12.78° or 90°, conductivity between 0.006 and 0.187 mS/cm and attenuation factor values between 0.12 and 1. Analyses were performed in triplicate, on two different production batches, transferring 1 mL of sample into a suitable cuvette for each one of them. No dilution was necessary as the count rate values of the events was within the acceptable limits of the instrument.

### Results of Example 1

### PSPA-L 17:1 Liposomes Enhance the Antimicrobial Response Against Mab

Liposomes consisting of PS and PA were generated with the classic lipid film hydration method (PSPA-L), and was assessed their efficacy in enhancing the intracellular killing of Mab. In particular, human macrophages were infected in vitro with Mab and then were stimulated with PSPA-L liposomal formulations having different molar ratios between PS and PA [Molar ratio PS:PA: 0.5:1; 1:1; 2:1 and 17:1 (FIG. 1A); 8.5:1, and 17:1 and 34:1 (FIG. 1 B)]. The results show that all formulations tested are able to significantly increase the intracellular killing of Mab and that the PSPA-L formulation having molar ratio 17:1 is particularly effective in intracellular killing of Mab (see FIGS. 1A and B).

The Addition of Cholesterol to the PSPA-L 17:1 Liposomal Formulation or its Dosage Variation does not Interfere with the Efficacy in Terms of Intracellular Killing of Mab

PSPA-L 17:1 liposomal formulations were generated with or without the addition of 30% of CHO (PSPACHO-L-Molar ratio 16.5:1:7.5) in order to analyze on macrophage cells infected with Mab the efficacy of the two formulations in determining the intracellular killing of Mab. Furthermore, it was assessed whether the treatment consisting of a double dose of liposomes (2×PSPA-L 17:1) could be reflected in an increase in intracellular mycobacterium killing. The results in FIG. 2 show no significant differences in the intracellular killing of Mab, highlighting that the cholesterol does not influence the biological activity of the liposomal formulations and that the latter is not dependent on the dosage of liposomes.

### The PSPA-L 17:1 or PSPACHO-L-Amikacin Combined Treatment Additively Reduces the Intracellular Growth of Mab in Human Macrophages

In order to establish the effect of a combined therapy based on antibiotics and bioactive liposomes, the in vitro treatment with PSPA-L 17:1 or PSPACHO-L and amikacin was tested on dTHP1 cells infected with Mab. The results show that PSPA-L 17:1 or PSPACHO-L reduce the intracellular replication of Mab in dTHP-1 cells (FIGS. 3A and B), without any direct effect on the extracellular pathogen (FIG. 3 C and D). It should be noted that the combined treatment with PSPA-L 17:1 or PSPACHO-L and AMK (FIGS. 3A and B) induced a significantly greater reduction in the Mab replication index with respect to the single treatments.

The Treatment with PSPA-L 17:1 and PSPACHO Liposomes does not Influence the Cell Viability of Human Macrophages

In order to assess whether the PSPA-L 17:1 and PSPACHO-L liposomal preparations, generated with the classical method of lipid film hydration, did not induce any toxic effect on human macrophages, the MTT cell viability assay was performed. The results shown in FIG. 4 show that there is no variation in cell viability following the treatment with PSPA-L (17:1) or PSPACHO-L confirming the in vitro non-toxicity of the liposomal formulations.

The Asymmetric PS/PA Liposomes and PSPA-L 17:1 Liposomes have a Different Distribution of PS on the Outer Leaflet of the Liposome

The distribution of phosphatidylserine of PSPA-L 17:1 and PSPACHO-L (liposomes according to the invention) and PS/PA liposomes was assessed.

In particular, a fluorescent analogue of PS (PS-NBD) and PA was employed and liposomal formulations were generated with the classical lipid film hydration method (PS-NBDPA-L and PS-NBDPACHO-L) or with the Weitz method (PS-NBD/PA). In order to assess the presence of PS-NBD on the inner surface of liposomes, these were exposed to a quenching solution capable of reducing the fluorescence only of PS-NBD localized on the outer leaflet of the liposome, not being able to cross its lipid bilayer. The results in FIG. 5A show a progressive decrease in fluorescence, which reaches almost total extinction, following the addition of the quencher to the PS-NBD/PA liposomes, validating the presence of phosphatidylserine on the outer leaflet (Proc Natl Acad Sci USA. 2012; 109(21):E1360-E1368). Whereas, in FIGS. 5 B and C, it is possible to note that the addition of the quencher to the liposomes leads to a partial decrease in fluorescence, although it is greater in the case of PS-NBDPACHO-L liposomes, indicating the presence of PS on both leaflets of the liposome and confirming then the structural diversity of the two liposomal formulations.

The Asymmetric PS/PA Liposomes and the PSPA-L 8.5:1 and PSPA-L 34:1 Liposomes have a Different Distribution of PS on the Outer Leaflet of the Liposome

The distribution of phosphatidylserine of PSPA-L 8.5:1 and 34:1 (liposomes according to the invention) and PS/PA liposomes was assessed using the same method as described above.

In particular, a fluorescent analogue of PS (PS-NBD) and PA was employed and liposomal formulations were generated with the classical lipid film hydration method (PS-NBDPA-L 8.5:1 and PS-NBDPA-L 34:1) which were compared with PS/PA liposomes generated with the Weitz method (PS-NBD/PA).

In order to assess the presence of PS-NBD on the inner surface of liposomes, these were exposed to a quenching solution capable of reducing the fluorescence only of PS-NBD localized on the outer leaflet of the liposome, not being able to cross its lipid bilayer.

The Applicant has noted that the addition of the quencher to the liposomes leads to a partial decrease in fluorescence with a trend similar to the one shown in FIG. 5 B and fluorescence plateau at around 0.7 (FIGS. 5 D and 5 E), indicating the presence of phosphatidylserine on both leaflets of the liposome and confirming then the structural diversity of the two liposomal formulations.

PSPA-L 8.5:1, PSPA-L 17:1, PSPA-L 34:1, and PSPACHO-L Liposomes have Different Chemical/Physical Characteristics Compared to Asymmetric PS/PA Liposomes.

Table 2 below indicates the zeta potential values, polydispersity index, and size values detected for PSPA-L 8.5:1, PSPA-L 17:1, PSPA-L 34:1, PSPACHO-L and PS/PA in samples of two batches of liposomes. For each batch, measurements were carried out in triplicate. Batch 1 and batch 2 relative to the same liposomal preparation were produced at different times and the different results for each batch depend on the variability of the laboratory method.

TABLE 2a
Batch 1

| | A | B | C | Average |
|---|---|---|---|---|
| PS/PA | | | | |
| Average-Z (nm) | 726.9 | 1680 | 1177 | 1194.6 ± 476.8 |
| Polydispersity index | 0.670 | 1.000 | 0.987 | 0.886 ± 0.187 |
| Zeta potential (mV) | −23.25 | −21.48 | −21.21 | −21.98 ± 1.108 |
| PSPA-L 8.5:1 | | | | |
| Average-Z (nm) | 84.72 | 73.24 | 74.72 | 77.56 ± 6.245 |
| Polydispersity index | 0.3241 | 0.3108 | 0.4874 | 0.3741 ± 0.098 |
| Zeta potential (mV) | −32.96 | −35.96 | −43.22 | −37.38 ± 5.275 |
| PSPA-L 17:1 | | | | |
| Average-Z (nm) | 118.8 | 120 | 118.8 | 119.2 ± 0.693 |
| Polydispersity index | 0.309 | 0.321 | 0.251 | 0.294 ± 0.037 |
| Zeta potential (mV) | −49.33 | −52.53 | −54.43 | −52.10 ± 2.577 |
| PSPA-L 34:1 | | | | |
| Average-Z (nm) | 173 | 177.2 | 171.9 | 174.03 ± 2.797 |
| Polydispersity index | 0.1391 | 0.1445 | 0.1319 | 0.1385 ± 0.006 |
| Zeta potential (mV) | −52.48 | −53.4 | −54.77 | −53.55 ± 1.152 |
| PSPACHO-L | | | | |
| Average-Z (nm) | 134.8 | 126.8 | 132 | 131.2 ± 4.06 |
| Polydispersity index | 0.353 | 0.338 | 0.353 | 0.348 ± 0.009 |
| Zeta potential (mV) | −60.34 | −62.79 | −61.16 | −61.43 ± 1.247 |

TABLE 2b
Batch 2

| | A | B | C | Average |
|---|---|---|---|---|
| PS/PA | | | | |
| Average-Z (nm) | 384.6 | 517.7 | 380.8 | 428.0 ± 77.96 |
| Polydispersity index | 0.465 | 0.608 | 0.389 | 0.490 ± 0.111 |
| Zeta potential (mV) | −18.22 | −17.24 | −18.08 | −18.00 ± 0.530 |
| PSPA-L 8.5:1 | | | | |
| Average-Z (nm) | 35.61 | 41.19 | 36.6 | 37.8 ± 2.977 |
| Polydispersity index | 0.2145 | 0.1473 | 0.2477 | 0.203 ± 0.0511 |
| Zeta potential (mV) | −53.7 | −51.65 | −52.23 | −52.52 ± 1.056 |
| PSPA-L 17:1 | | | | |
| Average-Z (nm) | 171.3 | 172.1 | 180.1 | 175.0 ± 4.87 |
| Polydispersity index | 0.217 | 0.265 | 0.385 | 0.290 ± 0.086 |
| Zeta potential (mV) | −33.75 | −34.19 | −34.69 | −34.00 ± 0.470 |
| PSPA-L 34:1 | | | | |
| Average-Z (nm) | 353.4 | 338.4 | 338.8 | 343.5 ± 8.547 |
| Polydispersity index | 0.518 | 0.5299 | 0.4767 | 0.5082 ± 0.0279 |
| Zeta potential (mV) | −35.22 | −37.45 | −36.16 | −36.27 ± 1.119 |
| PSPACHO-L | | | | |
| Average-Z (nm) | 199.5 | 188 | 187.4 | 192 ± 6.82 |
| Polydispersity index | 0.328 | 0.260 | 0.224 | 0.270 ± 0.052 |
| Zeta potential (mV) | −35.71 | −35.81 | −35.6 | −36.0 ± 0.105 |

### Polydispersity Index

The results obtained indicate that the PSPA-L 8.5:1, PSPA-L 17:1, PSPA-L 34:1 and PSPACHO-L preparations have a single peak corresponding to a single particle population, resulting more homogeneous with respect to the PS/PA preparation (FIG. 6 B-E and Table 2).

The results relative to the analysis of the PS/PA sample show the presence of three peaks corresponding to three different particle populations having different particle sizes that may be within the micrometer range (FIG. 6A). In fact, the liposomal formulation results to be highly inhomogeneous, having a polydispersity index of 0.8857±0.187, measurement detected in the batch 1, and of 0.49±0.111, measurement detected in the batch 2.

### Zeta Potential

The analysis of the zeta potential of the PSPA-L and PSPACHO-L liposomes shows values below −30 mV or −50 mV in the batch 2, and even below −60 mV in the batch 1, highlighting a high sample stability, due to an electrostatic repulsion suitable for achieving a better physical colloidal stability.

On the contrary, the results relative to the zeta potential of the PS/PA sample showed values between about −18 mV and about −22 mV, indicating then a low electrostatic repulsion associated with an instability of the sample that can cause aggregation and flocculation of the particles due to the attractive van der Waals forces acting on them.

### Sizes

The liposome size values indicated in table 2 are relative to the average Z size detected by DLS.

As it is known that the DLS technique is not the technique of choice for the analysis of samples with sizes approaching the micrometer, the measurement of the PS/PA sample was carried out in an ad hoc cuvette that allows the measurement of larger particles (up to 10 μm) with greater precision thanks to the intrinsic physical qualities of the capillary sizing cell, eliminating the errors associated with multiple scattering and also allowing the measurement of samples over a wider dynamic concentration range than would normally be possible at lateral dispersion (90°). The PSPA-L 8.5:1, PSPA-L 17:1, PSPA-L 34:1 and PSPACHO-L liposomes result to have smaller sizes than the asymmetric PS/PA liposomes.

The data obtained show a significant difference between the PSPA-L 8.5:1, PSPA-L 17:1, PSPA-L 34:1 and PSPACHO-L liposomes and the asymmetric PS/PA liposome, both in terms of size and zeta potential values, indicating that the preparation method adopted leads to the obtaining of two different products in terms of chemical-physical characteristics and consequently stability and homogeneity. Furthermore, the PSPA-L 8.5:1, PSPA-L 17:1, PSPA-L 34:1 and PSPACHO-L liposomes produced with the classical lipid film hydration method, although not having identical lipid composition, have very similar distribution characteristics.

### Example 2: Microfluidic Hydrodynamic Focusing Technique

### Materials and Methods

### Production of Liposomes PSPA-M 17:1

In the example 2, the abbreviation “PSPA-M” indicates liposomes produced through the microfluidic hydrodynamic focusing technique comprising PS on the outer leaflet and PS and PA on the inner leaflet.

For the production of these liposomes, L-α-phosphatidylserine (PS; Avanti Polar Lipids) and L-α-phosphatidic acid (PA; Avanti Polar Lipids) were initially reconstituted in methanol at a concentration of 10 mg/ml and 1 mg/ml, respectively. In order to obtain 1 ml of final solution with a total lipid concentration (PS+PA) of 5 mg/ml wherein the molar ratio between PS and PA was kept at 17:1, 496 μl of PS, 238 μl of PA were taken and 267 μl of methanol was added.

This solution was used as the alcohol phase in the Dolomite microfluidic system, while double-distilled water was used for the aqueous phase. The flow rate ratio (FRR) between the two solutions was set at 7.5 (300 μl/min aqueous solution, 40 μl/min lipid solution). The resulting liposomes (5 ml final solution) were collected in a 5 ml tube and half of the solution was dialyzed for 18 hours in one liter of double-distilled water using PUR-A-LYZER MAXI 6000 dialysis membranes (Molecular Weight Cut-Off 6-8 kDa).

### Cell Cultures

dTHP-1

The monocyte/macrophage cell line THP-1 was provided by the European Collection of Authenticated Cell Culture (ECACC 88081201), and cultured in RPMI 1640 containing fetal bovine serum (10%), gentamicin (5 μg/ml), L-glutamine (2 mM), non-essential acidic amino acids (1 mM), sodium pyruvate (1 mM) in 75 cm² polystyrene flasks. Before the experiments, the cells (5×10⁵ or 2×10⁵) were seeded in 24 or 96 well-plates and the cells were induced to differentiate through stimulation for 72 hours with forbol-12-myristate 13-acetate (PMA) (20 ng/ml) and used as a model of human macrophages (dTHP-1).

### Macrophages of Type 1 (M1) or of Type 2 (M2)

The peripheral blood mononuclear cells (PBMCs) from healthy donors were isolated using the Ficoll density gradient. Monocytes were then positively selected using anti-CD14 monoclonal antibodies conjugated to magnetic microbeads (Miltenyi Biotec) according to the manufacturer's instructions. Monocytes (5×10⁵ or 2×10⁵) were seeded in 24 or 96 well-plates and the cells were induced to differentiate in the presence of granulocyte-macrophage colony-stimulating factor (GM-CSF) (35 ng/mL) or a macrophage colony stimulating factor (M-CSF) (50 ng/ml) (R&D Systems) in order to obtain type 1 (M1) or type 2 (M2) macrophages, respectively.

### Bacterial Cultures

The individual colonies of Mycobacterium abscessus (Mab) (ATCC® 19977) were obtained by seeding the mycobacterium on Middlebrook 7H10 (7H10-BD Difco™) solid medium integrated with oleic acid, albumin, dextrose and catalase (OADC), and then resuspended in 15 ml of Middlebrook 7H9 broth (7H9-BD Difco™) integrated with albumin, dextrose and catalase (ADC) and grown in Erlenmeyer flask at 37° C. in agitation for 48 hours. The growth of bacterial cultures was monitored by measuring the optical density at a wavelength of 600 nm with a spectrophotometer (Varioskan LUX Multimode Microplate Reader, Thermo Fisher Scientific). The bacilli were stored at −80° C. until use after suspension in Microorganism Preservation System-Protect (Technical Service Consultants Ltd).

MTT Assay for Assessment of dTHP-1 Cell Viability

The cell viability of dTHP-1, previously stimulated for 18 hours or 5 days with PSPA-M 17:1 liposomes produced with the microfluidic hydrodynamic focusing technique undergoing or not undergoing dialysis, whose phospholipids concentration was equal to 2.54 μM, 1.27 μM, or 0.635 μM was assessed by assay with 3-(4,5-dimethylthiazol-2yl)-2,5-diphenyl tetrazolium bromide (MTT). The assay with MTT (Molecolar Probe) is based on the intracellular reduction of tetrazolium salts by the dehydrogenase succinate mitochondrial enzyme (SDH) into crystals of a bluish product called formazan, a reduction that can only be produced in metabolically active cells whose optical density is measured by spectrophotometer using a wavelength of 540 nm. This assay was conducted according to the guidelines indicated in the user manual.

### MTT Assay for Assessment of M1 and M2 Macrophages Cell Viability

The cell viability of M1 and M2 macrophages, previously stimulated for 18 hours or 5 days with PSPA-M 17:1 liposomes produced with the microfluidic hydrodynamic focusing technique undergoing or not undergoing dialysis, whose phospholipids concentration was equal to 2.54 μM, 1.27 μM, or 0.635 μM was assessed by assay with 3-(4,5-dimethylthiazol-2yl)-2,5-diphenyl tetrazolium bromide (MTT). The assay with MTT (Molecolar Probe) is based on the intracellular reduction of tetrazolium salts by the dehydrogenase succinate mitochondrial enzyme (SDH) into crystals of a bluish product called formazan, a reduction that can only be produced in metabolically active cells whose optical density is measured by spectrophotometer using a wavelength of 540 nm. This assay was conducted according to the guidelines indicated in the user manual.

### Infection and Assessment of In Vitro Bacterial Intracellular Growth-dTHP-1 Cells

In order to assess the intracellular bacterial growth, dTHP-1 cells were distributed in 24 well-plates at the concentration of 5×10⁵ cells/ml. The cells were infected with Mab, for 3 hours at 37° C. at a MOI of 10, and after the infection the extracellular bacilli were killed through a 1-hour incubation with 250 μg/ml of amikacin. Cells were then washed and incubated for further 18 hours with PSPA-M 17:1 liposomes produced with the microfluidic hydrodynamic focusing technique undergoing or not undergoing dialysis, whose phospholipids concentration was equal to 1.27 μM. Finally, the cells were lysed with 1% deoxycholate (Sigma), samples diluted in PBS-tween 80 and CFUs were quantified by plating the bacilli in triplicate on solid Middlebrook 7H10 Agar growth medium.

### Infection and Assessment of In Vitro Bacterial Intracellular Growth—M1 and M2 Macrophages

In order to assess the intracellular bacterial growth, M1 and M2 were distributed in 24 well-plates at the concentration of 5×10⁵ cells/ml. The cells were infected with Mab, for 3 hours at 37° C. at a MOI of 10, and after the infection the extracellular bacilli were killed by a 1-hour incubation with 250 μg/ml of amikacin. Cells were then washed and incubated for further 18 hours with PSPA-M 17:1 liposomes produced with the microfluidic hydrodynamic focusing technique undergoing dialysis, whose phospholipids concentration was equal to 1.27 μM. Finally, the cells were lysed with 1% deoxycholate (Sigma), samples diluted in PBS-tween 80 and CFUs were quantified by plating the bacilli in triplicate on solid Middlebrook 7H10 Agar growth medium.

### Distribution of Phosphatidylserine on the Surface of Liposomes

The distribution of phosphatidylserine of PSPA-M 17:1, dialyzed PSPA-M 17:1 and PSPA-L17:1 liposomes was assessed.

In particular, it was used a fluorescent analogue of PS (PS-NBD) and PA and were generated liposomal formulations with the classic lipid film hydration method (PS-NBDPA-L 17:1) or the microfluidic hydrodynamic focusing technique (PS-NBDPA-M 17:1 and dialyzed PS-NBDPA-M 17:1). In order to assess the presence of PS-NBD on the inner surface of liposomes, these were exposed to a quenching solution capable of reducing the fluorescence only of PS-NBD localized on the outer leaflet of the liposome, not being able to cross its lipid double layer.

Fluorescence measurement was performed in kinetics for 5 min every 30 seconds (12 measurements) with the Varioskan LUX multimode microplate reader fluorometer (Thermo Fisher Scientific) by setting the excitation and emission wavelengths to 470 nm and 550 nm respectively.

### DLS Characterization of Liposomes

The experiments of characterization of PSPA-M 17:1 liposomes undergoing or not undergoing dialysis were carried out on samples stored in a refrigerator at 4° C., using the Zeta-Sizer Advance Ultra-Malvern Panalytical instrument, provided with a He—Ne laser (λ=633 nm) with a maximum power of 10 mW. Measurements were carried out at a constant temperature value of 25° C., with a scattering angle of 12.78° or 90°, conductivity between 0.006 and 0.187 mS/cm and attenuation factor values between 0.12 and 1. Analyses were performed in triplicate, on two different production batches, transferring 1 mL of sample into a suitable cuvette for each one of them. No dilution was necessary as the count rate values of the events were within the acceptable limits of the instrument.

### Results of Example 2

The Treatment with Dialyzed PSPA-M 17:1 or with PSPA-M 17:1 Liposomes does not Influence the Cell Viability of dTHP-1 Human Macrophages

Cell viability assays highlighted that liposomes produced with the microfluidic hydrodynamic focusing technique, whether dialyzed or not, do not reduce the cell viability of dTHP-1 human macrophages after 18 hours or 5 days of stimulation, indicating their non-toxicity (see FIGS. 7A and B).

The Treatment with Dialyzed PSPA-M 17:1 or with PSPA-M 17:1 Liposomes does not Influence the Cell Viability of Primary M1 or M2 Macrophages

Cell viability assays highlighted that liposomes produced with the microfluidic hydrodynamic focusing technique, whether dialyzed or not, do not reduce the cell viability of primary M1 or M2 macrophages after 18 hours or 5 days of stimulation, indicating their non-toxicity (see FIG. 10).

### PSPA-M 17:1 Liposomes Enhance the Antimicrobial Response Against Mab

The results obtained on dTHP-1 human macrophages show that liposomes produced with the microfluidic hydrodynamic focusing technique are able to significantly increase the intracellular killing of the mycobacterium. Furthermore, it can be noted that the dialysis process does not interfere with the bioactivity of the formulation (see FIG. 8).

### Dialyzed PSPA-M 17:1 Liposomes Enhance the Antimicrobial Response Against Mab in Primary M1 or M2 Macrophages

The results previously obtained on dTHP-1 human macrophages highlighted that there is no significant difference in the biological effect between PSPA-M 17:1 liposomes and dialyzed PSPA-M 17:1. Furthermore, dialyzed PSPA-M 17:1 liposomes did not prove to be toxic following MTT assays on primary M1 or M2 macrophages and their physical-chemical characterization showed how they are more stable with respect to non-dialyzed PSPA-M 17:1 liposomes in terms of zeta potential. For these reasons, and considering also that, for liposomes produced with the MHF technique, the dialysis process is a key step for the removal of any residual traces of organic solvents, exclusively dialyzed PSPA-M 17:1 liposomes were selected for subsequent bioactivity tests on primary M1 or M2 macrophages.

The results reported in FIG. 11 show that dialyzed PSPA-M 17:1 liposomes are able to significantly increase the intracellular killing of the mycobacterium.

PSPA-M 17:1, Dialyzed PSPA-M 17:1 and PSPA-L 17:1 Liposomes have the Same Distribution of PS on the Outer Leaflet of the Liposome

The results in FIG. 9 show that the addition of quencher to the liposomes leads to a partial decrease in fluorescence, indicating the presence of PS on both leaflets of the liposome and demonstrating then the structural similarity of the three liposomal formulations.

### Homogeneity, Stability and Sizes of PSPA-M 17:1 Liposomes

Tables 3a and 3b below indicate the average zeta potential, polydispersity index, and sizes values of PSPA-M 17:1 liposomes undergoing or not undergoing dialysis detected in two batches.

TABLE 3a
Batch 1

| | No dialysis | Dialysis |
|---|---|---|
| Average-Z (nm) | 239.6 ± 2.745 | 175 ± 2.124 |
| Polydispersity index | 0.1276 ± 0.061 | 0.1246 ± 0.023 |
| Zeta potential (mV) | −55.26 ± 1.632 | −73.31 ± 2.342 |

TABLE 3b
Batch 2

| | No dialysis | Dialysis |
|---|---|---|
| Average -Z (nm) | 285.9 ± 7.881 | 161 ± 1.331 |
| Polydispersity index | 0.1765 ± 0.02 | 0.1789 ± 0.023 |
| Zeta potential (mV) | −56.81 ± 1.429 | −81.6 ± 0.4547 |

The characterization in terms of size, polydispersity index and zeta potential of the liposomes produced with the microfluidic hydrodynamic focusing technique highlighted a greater homogeneity with respect to the liposomes generated with the lipid film hydration method. The Applicant has furthermore advantageously noted that the dialysis process has an effect on the liposomes: these become smaller and their zeta potential decreases, but there are no differences in terms of the polydispersity index.

1. A liposome consisting of an outer leaflet comprising phosphatidylserine, and an inner leaflet comprising phosphatidylserine and phosphatidic acid, wherein zeta potential of said liposome is between −25 mV and −100 mV and molar ratio between the total phosphatidylserine comprised on the outer and on the inner leaflet and the phosphatidic acid is between 5:1 and 35:1.

2. The liposome according to claim 1, wherein the molar ratio between the phosphatidylserine comprised on the outer and on the inner leaflet and the phosphatidic acid is between 16:1 and 18:1.

3. The liposome according to claim 1, wherein the molar ratio between the phosphatidylserine comprised on the outer and on the inner leaflet, and the phosphatidic acid is 17:1.

4. The liposome according to claim 1, wherein the zeta potential of said liposome is between −25 mV and −85 mV.

5. The liposome according to claim 1, further comprising cholesterol, said cholesterol being present on the outer leaflet and/or on the inner leaflet of the liposome.

6. The liposome according to claim 5, wherein the molar ratio between said phosphatidylserine comprised on the outer and on the inner leaflet, said phosphatidic acid, and said cholesterol is 16.5:1:17.5.

7. The liposome according to claim 1, which comprises at least one first antibiotic therein.

8. The liposome according to claim 7, wherein said at least one first antibiotic is selected from first-line antitubercular antibiotics; second-line antitubercular antibiotics; aminoglycosides; glycylcyclines; penicillins; carbapenems; monobactams; quinolones; oxazolidinones; and macrolides.

9. A pharmaceutical composition comprising at least one liposome according to claim 8, and at least one pharmaceutically acceptable excipient.

10. The pharmaceutical composition according to claim 9, further comprising at least one second antibiotics positioned outside said liposome, wherein said at least one second antibiotics is selected from first-line antitubercular antibiotics, second-line antitubercular antibiotics, glycylcyclines, tetracyclines, cephalosporins, penicillins, carbapenems, monobactams, quinolones, oxazolidinone, macrolides.

11-16. (canceled)

17. A method for the treatment of one or more bacterial infections in a subject in need thereof, said method comprising
administering to said subject having one or more bacterial infections one or more of the liposomes according to claim 8 and/or a pharmaceutical composition comprising said one or more liposomes, and at least one pharmaceutically acceptable excipient.

18. The method according to claim 17, wherein said one or more liposomes and/or said pharmaceutical composition are administered by inhalation.

19. The method according to claim 17, wherein said bacterial infections are caused by Pseudomonas aeruginosa, Enterococcus faecium, Staphylococcus aureus, Klebsiella pneumoniae, Acinetobacter baumannii, Enterobacter spp, Propionibacterium acnes, Mycobacterium tuberculosis, Mycobacterium africanum, Mycobacterium bovis, Mycobacterium ulcerans, Mycobacterium leprae, Mycobacterium chimaera, Mycobacterium kansasii, Mycobacterium tuberculosis complex, Mycobacterium avium complex (MAC), and M. abscessus complex.

20. The method according to claim 17, wherein said infections are caused by mycobacteria micobacteria.

21. The method according to claim 20, wherein said micobacteria are nontuberculous mycobacteria micobacteria.

22. The method according to claim 21, wherein said nontuberculous mycobacteria micobacteria are selected from Mycobacterium avium complex (MAC), Mycobacterium chimaera, Mycobacterium kansasii, and M. abscessus complex.

23. The method according to claim 22, wherein the M. abscessus complex comprises M. abscessus ssp. abscessus, M. abscessus ssp. bolletii and M. abscessus ssp. massiliense.

24. The method according to claim 17, wherein said bacteria infections are infections caused by Mycobacterium abscessus spp.

25. The liposome according to claim 4, wherein the zeta potential of said liposome is between −30 mV and −75 mV.

26. The liposome according to claim 4, wherein the zeta potential of said liposome is between −35 mV and −50 mV.
